(19) United States
(12) Patent Application Publication
(10) Pub. No.: US 2026/0224495 A1
(43) Pub. Date: Aug. 6, 2026

(54) OSMOTIC PHARMACEUTICAL CAPSULE

(71) Applicant: R.P. SCHERER TECHNOLOGIES, LLC, Carson City, NV (US)

(72) Inventors: Lester David Fulper, Clearwater, FL (US); Zahra Karjoo, Pinellas Park, FL (US)

(21) Appl. No.: 19/152,988

(22) PCT Filed: Feb. 2, 2024
(86) PCT No.: PCT/US2024/014246

§ 371 Date: Aug. 1, 2025

Related U.S. Application Data
(60) Provisional application No. 63/443,142, filed on Feb. 3, 2023.

(51) Int. Cl. A61K 9/48; A61K 31/167

(52) U.S. Cl. CPC A61K 9/4808; A61K 9/4816; A61K 9/4825; A61K 9/4833; A61K 9/4858; A61K 9/4866; A61K 31/167

(57) ABSTRACT

Disclosed in certain embodiments is a capsule comprising a liquid fill material comprising an osmogen and an active agent; and a semi-permeable disintegration resistant shell composition.

### CROSS REFERENCE TO RELATED APPLICATION(S)

The present application claims priority to U.S. Provisional Patent Application No. 63/443,142 filed on Feb. 3, 2023, the entire contents of which are incorporated in its entirety.

### FIELD OF THE INVENTION

The present invention relates to capsules, (e.g., softgels) including a liquid fill material comprising an osmogen and an active agent; and a semi-permeable disintegration resistant shell composition.

### BACKGROUND OF THE INVENTION

Capsules, in particular, hard or soft gelatin capsules (or softgel capsules), provide a dosage form which is more readily accepted by patients, since the capsules are easy to swallow and need not be flavored in order to mask any unpleasant taste of the active agent. Softgel encapsulation of drugs further provides the potential to improve the bioavailability of the pharmaceutical agents. For example, active ingredients may be rapidly released in liquid form as soon as the gelatin shell ruptures.

There are a number of osmotic delivery systems available that work on the principle of osmotic pressure across a semipermeable membrane forcing the active out of the device through pores or purposely placed holes. These osmotic delivery systems may provide clinical benefits such as controlled release of active agent, minimization of peak to trough” fluctuation, reduction in dosing, reduced dosage frequency, reduced side effects, and improved patient compliance.

There exists a need in the art for a drug delivery system that provided combines the benefits of both liquid filled capsules and osmotic delivery systems.

### OBJECTS AND SUMMARY OF THE INVENTION

It is an object of certain embodiments of the present invention to provide an osmotic capsule capable of delivery of a liquid payload.

It is an object of further embodiments of the invention to provide methods of preparing the osmotic capsules as disclosed herein.

It is an object of further embodiments of the invention to provide methods of treating a disease or condition comprising orally administering to a patient in need thereof an osmotic capsule as disclosed herein.

It is an object of further embodiments of the invention to provide a device capable of piercing an osmotic capsules as disclosed herein prior to ingestion by a patient.

One or more of the above objects and others, are achieved by the present invention which in certain embodiments are directed to capsule comprising a liquid fill material comprising an osmogen and an active agent; and a semi-permeable disintegration resistant shell composition. In certain embodiments, the capsule has an orifice through the shell that is formed at the time of manufacture or by the patient or caregiver prior to administration.

### BRIEF DESCRIPTION OF THE DRAWINGS

FIG. 1 shows a progression of a liquid filled capsule in an embodiment of the present invention, being pierced by a stylus and then removed, with the pierced capsule placed in water.

FIG. 2 shows a plurality of capsules in a piercing drug delivery device in a carousel configuration.

FIGS. 3a-3c shows a cross section of a capsule according to a variety of embodiments of the present disclosure.

FIG. 4 shows a preparation of a liquid filled capsule according to another embodiment of the present invention.

FIG. 5 shows the impact of crosslinking level and hole size relative to release profile.

### DETAILED DESCRIPTION OF THE INVENTION

The present invention advances the state of the art by developing an osmotic dosage form capable of delivering a liquid payload. It combines the convenience of pharmaceutical capsules (e.g. softgels) with the clinical benefits of osmotic dosage forms. In certain embodiments, capsule shell is semipermeable to provide for the influx of water due to the presence of the osmogen in the liquid fill, whereby the resultant pressure gradient causes the drug to exit the capsule through an orifice and/or through pores formed in the shell and/or by controlled rupture of the capsule.

Certain embodiments of the present invention address the potential problem of leakage of the fill material from the orifice during transport and/or storage of the capsule post-manufacture. In certain embodiments, the orifice is created by the patient or caregiver just prior to administration (e.g., with a stylus or by a device that punctures the capsule upon actuation). In other embodiments, the orifice is made at the point of manufacture plugged or enrobed with a material that erodes after administration to expose the orifice to the liquid environment of the gastro-intestinal tract. In some embodiments, the orifice may be created by puncturing the capsule using a laser, stylus or drill. The orifice of the capsule may then be sealed using a piezeoelectric spray, which in turn may plug the orifice. In some embodiments, the capsule may be sealed using any suitable method to seal. The capsule may then further be enrobed with a soluble coating. In some embodiments, the orifice may be created by puncturing and then may further be enrobed with a soluble coating. In other embodiments, the orifice may be created by puncturing and is not sealed or enrobed.

In certain embodiments, the capsule shell is insoluble in the gastric and intestinal environment or at least resistant to disintegration for a length of time required to deliver the proposed dose of active agent. The shell therefore in certain embodiments comprises insoluble or disintegration resistant materials, or could be coated with a substance that provides this performance. The disintegration resistant shell can also comprise crosslinked gelatin or other polymers such as alginate or carrageenan. Such crosslinking processes can be performed, e.g., after filling the liquid fill into the capsule, or by adding crosslinking additives to the gel-mass prior to formation of the capsules whereby the crosslinking agent should have a reaction time delay sufficient to allow encapsulation before excessive cross linking would occur that would render the gel-mass un-machinable. In certain embodiments, in-line blending of the crosslinking agent with the gel-mass just prior to casting on the drum is one way to prepare the capsules disclosed herein. In certain embodiments, the insoluble or disintegration resistant material may include a basic resistant polymer.

The release rate of the active agent may be controlled by composition of the fill, composition of the shell, size of the orifice, or combinations of any or all of these factors. In certain embodiments, the fill material is hypoosmotic which will cause water to migrate into the capsule, thereby displacing the contents of the capsule though the orifice resulting in a period of relatively constant active agent delivery which will slowly taper as the contents of the capsule become diluted.

In certain embodiments, fill materials that are not hypoosmotic such as oils may be able to be modified by suspension of compatible salts or inclusion of water swelling materials. In such an embodiment, the immiscible fill material will not be diluted when water migrates into the interior of the capsule.

In certain embodiments the present invention is directed to a capsule comprising a liquid fill material comprising an osmogen and an active agent; and a semi-permeable disintegration resistant shell composition.

In certain embodiments, the shell comprises a film forming substance and a disintegration resistant material. In certain embodiments, the shell comprises a film forming substance, a disintegration resistant material, or a combination thereof. In certain embodiments, a film forming substance and a disintegration resistant material may include the same component.

In certain embodiments, the film forming substance and the disintegration resistant material are inter-dispersed.

In certain embodiments, the shell comprises an inner layer comprising the film forming substance and an outer layer comprising the disintegration resistant material. In some embodiments, the inner layer is understood as being a soluble layer, and the outer layer is understood as being an insoluble layer.

In some embodiments, the disintegration resistant material may include gums, cellulose ethers, acrylic resins, protein derived materials, waxes, shellac, and oils such as hydrogenated castor oil and hydrogenated vegetable oil. Other polymers include alkylcelluloses such as ethylcellulose, acrylic and methacrylic acid polymers and copolymers; and cellulose ethers, such as hydroxyalkylcelluloses (e.g., hydroxypropylmethylcellulose) and carboxyalkylcelluloses. Other acrylic and methacrylic acid polymers and copolymers include methyl methacrylate, methyl methacrylate copolymers, ethoxyethyl methacrylates, ethyl acrylate, trimethyl ammonioethyl methacrylate, cyanoethyl methacrylate, aminoalkyl methacrylate copolymer, poly(acrylic acid), poly(methacrylic acid), methacrylic acid alkylamine copolymer, poly(methylmethacrylate), poly(methacrylicacid) (anhydride), polymethacrylate, polyacrylamide, poly(methacrylic acid anhydride), glycidyl methacrylate copolymers and combinations thereof. Acrylic polymers useful as disintegration resistant materials include acrylic resins comprising copolymers synthesized from acrylic and methacrylic acid esters (e.g., the copolymer of acrylic acid lower alkyl ester and methacrylic acid lower alkyl ester) containing about 0.02 to 0.03 mole of a tri (lower alkyl) ammonium group per mole of the acrylic and methacrylic monomers used. An example of a suitable acrylic resin is a polymer manufactured by Rohm Pharma GmbH and sold under the Eudragit® RS trademark. Eudragit RS30D may be used. Eudragit® RS is a water insoluble copolymer of ethyl acrylate (EA), methyl methacrylate (MM) and trimethylammoniumethyl methacrylate chloride (TAM) in which the molar ratio of TAM to the remaining components (EA and MM) is 1:40. Acrylic resins such as Eudragit® RS may be used in the form of an aqueous suspension.

In some embodiments, the disintegration resistant material may include a cellulose polymer selected from the group consisting of ethylcellulose, cellulose acetate, cellulose propionate (lower, medium or higher molecular weight), cellulose acetate propionate, cellulose acetate butyrate, cellulose acetate phthalate and cellulose triacetate. An example of ethylcellulose is one that has an ethoxy content of 44 to 55%. Ethylcellulose may be used in the form of an alcoholic solution. In certain other embodiments, the hydrophobic material comprises polylactic acid, polyglycolic acid or a co-polymer of the polylactic and polyglycolic acid.

In certain embodiments, the disintegration resistant material may include a cellulose polymer selected from the group consisting of cellulose ether, cellulose ester, cellulose ester ether, and cellulose. The cellulosic polymers may have a degree of substitution, D.S., on the anhydroglucose unit, from greater than zero and up to 3 inclusive. Representative materials include a polymer selected from the group consisting of cellulose acylate, cellulose diacylate, cellulose triacylate, cellulose acetate, cellulose diacetate, cellulose triacetate, mono, di, and tricellulose alkanylates, mono, di, and tricellulose aroylates, and mono, di, and tricellulose alkenylates. Exemplary polymers include cellulose acetate having a D.S. and an acetyl content up to 21%; cellulose acetate having an acetyl content up to 32 to 39.8%; cellulose acetate having a D.S. of 1 to 2 and an acetyl content of 21 to 35%; cellulose acetate having a D.S. of 2 to 3 and an acetyl content of 35 to 44.8%.

Other cellulosic polymers include cellulose propionate having a D.S. of 1.8 and a propyl content of 39.2 to 45 and a hydroxyl content of 2.8 to 5.4%; cellulose acetate butyrate having a D.S. of 1.8, an acetyl content of 13 to 15% and a butyryl content of 34 to 39%; cellulose acetate butyrate having an acetyl content of 2 to 29%, a butyryl content of 17 to 53% and a hydroxyl content of 0.5 to 4.7%; cellulose triacylate having a D.S. of 2.9 to 3 such as cellulose triacetate, cellulose trivalerate, cellulose trilaurate, cellulose tripalmitate, cellulose trisuccinate, and cellulose trioctanoate; cellulose diacylates having a D.S. of 2.2 to 2.6 such as cellulose disuccinate, cellulose dipalmitate, cellulose dioctanoate, cellulose dipentanoate, and coesters of cellulose such as cellulose acetate butyrate, cellulose acetate octanoate butyrate and cellulose acetate propionate.

Additional cellulose polymers useful as disintegration resistant materials include acetaldehyde dimethyl cellulose acetate, cellulose acetate ethylcarbamate, cellulose acetate methylcarbamate, and cellulose acetate dimethylaminocellulose acetate.

In some embodiments, the disintegration resistant material and/or film forming material may include an osmotic polymer. Examples of osmotic polymers include, but are not limited to, poly (hydroxy-alkyl methacrylate) having a molecular weight of from 30,000 to 5,000,000; polyvinylpyrrolidone (PVP) having a molecular weight of from 10,000 to 360,000; anionic and cationic hydrogels; polyelectrolytes complexes; polyvinyl alcohol having a low acetate residual, cross-linked with glyoxal, formaldehyde, or glutaraldehyde and having a degree of polymerization of from 200 to 30,000; a mixture of methyl cellulose, cross-linked agar and carboxymethyl cellulose; a mixture of hydroxypropyl methylcellulose and sodium carboxymethylcellulose; a mixture of hydroxypropyl ethylcellulose and sodium carboxymethyl cellulose; sodium carboxymethylcellulose; potassium carboxymethylcellulose; a water insoluble, water swellable copolymer formed from a dispersion of finely divided copolymer of maleic anhydride with styrene, ethylene, propylene, butylene or isobutylene cross-linked with from 0.001 to about 0.5 moles of saturated cross-linking agent per mole of maleic anhydride per copolymer; water swellable polymers of N-vinyl lactams; polyoxyethylene-polyoxypropylene gel; polyoxybutylene-polyethylene block copolymer gel; carob gum; polyacrylic gel; polyester gel; polyuria gel; polyether gel; polyamide gel; polypeptide gels; polyamino acid gels; polycellulosic gel; polygum gel; and initially dry hydrogels that imbibe and absorb water that penetrates the glassy hydrogel and lowers its glass temperature.

Other examples of osmotic polymers include, but are not limited to, the following: polymers that form hydrogels such as CARBOPOL® (Noveon, Inc., Cleveland Ohio), acidic carboxypolymer, a polymer of acrylic and cross-linked with a polyallyl sucrose, also known as carboxypolymethylene and carboxyvinyl polymer having a molecular weight of 250,000 to 4,000,000; cynamer polyacrylamides; cross-linked water swellable indene-maleic anhydride polymers; GOOD-RITE® (Noveon, Inc., Cleveland Ohio) polyacrylic acid having a molecular weight of 80,000 to 200,000; POLYOX® (Union Carbide Chemicals & Plastics Technology Corporation, Danbury Conn.) polyethylene oxide polymer having a molecular weight of 100,000 to 5,000,000 and higher; starch graft copolymers; acrylate polymer polysaccharides composed of condensed glucose units such as diester cross-linked polygluran; and the like.

The osmogen may include an osmotic effective solute. The osmotic effective solute may include inorganic and organic compounds that can exhibit an osmotic pressure gradient across the semipermeable membrane when the osmotic delivery system is placed in a fluid environment. Osmotic effective solutes or osmogents (i.e., the non-volatile species that are soluble in water and create the osmotic gradient driving the osmotic inflow of water) useful in the osmotic agent formulation include, but are not limited to, magnesium sulfate, magnesium chloride, sodium chloride, potassium sulfate, sodium sulfate, lithium sulfate, sodium phosphate, potassium phosphate, d-mannitol, urea, inositol, magnesium succinate, tartaric acid, inositol, carbohydrates, and various monosaccharides, oligosaccharides and polysaccharides such as sucrose, glucose, lactose, fructose, raffinose and dextran, as well as mixtures of any of these various species.

Osmotic agents such as sodium chloride (NaCl) with appropriate excipients (lubricants and binders; e.g., cellulosic and povidone binders) and viscosity modifying agents, such as sodium carboxymethylcellulose or sodium polyacrylate are examples of preferred osmotic agents. Other osmotic agents useful as a water-swellable agent include osmopolymers and osmagents and are described, for example, in U.S. Pat. No. 5,413,572.

In certain embodiments, the shell composition includes a disintegration resistant material in an amount of from about 1%, about 5%, about 10%, about 15%, about 20%, about 25%, about 30%, about 35%, about 40%, about 45%, about 50%, about 55%, about 60%, about 70%, about 75%, about 80%, about 85%, about 90%, or to about 90%, about 85%, about 80%, about 75%, about 70%, about 65%, about 60%, about 55%, about 50%, about 45%, about 40%, about 35%, about 30%, about 25%, about 20%, about 15%, or about 10%, or any range or sub-value herein.

In certain embodiments, the shell composition includes a film forming material in an amount of from about 1%, about 5%, about 10%, about 15%, about 20%, about 25%, about 30%, about 35%, about 40%, about 45%, about 50%, about 55%, about 60%, about 70%, about 75%, about 80%, about 85%, about 90%, or to about 90%, about 85%, about 80%, about 75%, about 70%, about 65%, about 60%, about 55%, about 50%, about 45%, about 40%, about 35%, about 30%, about 25%, about 20%, about 15%, or about 10%, or any range or sub-value herein.

In certain embodiments, the capsule is a hard capsule, e.g., a two piece capsule.

In certain embodiments, the capsule is a softgel capsule.

In certain embodiments, the semi-permeable shell composition is permeable to the passage of water and impervious to the passage of the active agent.

In certain embodiments, the semi-permeable shell composition comprises at least one orifice. In certain embodiments, the semi-permeable shell composition comprises one orifice, two orifices, three orifices, four orifices, or five orifices, or more.

In certain embodiments, by imbibing fluid, such as water, through the shell composition into the capsule a pressure gradient is created that causes the active agent to expel from the orifice. The active agent expels from the orifice through the fill composition of the capsule at a slow and steady rate. In certain embodiments, if water goes through the shell composition to release the fill composition, then the fill composition becomes diluted as the active agent is released from the capsule. In certain embodiments, the pressure gradient and release rate of the fill composition can be controlled by the size of the orifice and the amount of orifices present in the shell composition. In certain embodiments, the size of the orifice may be about 600 μm to about 1 mm, about 625 μm to about 975 μm, about 650 μm to about 950 μm, about 675 μm to about 925 μm, about 700 μm to about 900 μm, about 725 μm to about 875 μm, about 750 μm to about 850 μm, or about 775 μm to about 825 μm.

In certain embodiments, the capsule further comprises a soluble plug filling or covering the orifice. The soluble plug filling may be made of a material that is soluble in water and will dissolve when contacted with water.

In certain embodiments, the capsule further comprises an additional soluble coating enrobing the shell composition and the orifice. The additional soluble coating may be made of a material that is soluble in water and will dissolve when contacted with water.

In certain embodiments, the capsule further comprises an expanding component in the capsule adjacent to the fill material.

In certain embodiments, the capsule further comprises an expanding component layered around the fill material.

In certain embodiments, the fill material is adjacent to an orifice.

In certain embodiments, an osmotic gradient causes water to be imbibed through the shell composition to cause the expanding component to expand and displace the active agent through the orifice.

In certain embodiments, the shell composition further comprises a pore former. The pore-former may be organic or inorganic, and include materials that can be dissolved, extracted or leached from the shell in the environment of use. The pore-formers may comprise a cellulosic material, e.g., hydroxypropylmethylcellulose; a polyalkylene glycol, e.g., polyethylene glycol; povidone; or a combination of any of the foregoing. In some embodiments, the pore former may include an alkaline metal salt, such as sodium chloride, sodium bromide, potassium chloride, potassium sulphate, or potassium phosphate, an alkaline earth metal, such as calcium chloride, or calcium nitrate, a carbohydrate, such as sucrose, glucose, fructose, mannose, lactose, sorbitol, or mannitol, and diols or polyols, such as polyhydric alcohols, polyethylene glycol, or polyvinyl pyrrolidone.

In certain embodiments, the pore former is soluble at a selected pH of the gastro-intestinal system.

In certain embodiments, an osmotic gradient causes water to be imbibed through the shell composition to cause the expanding component to expand and displace the active agent through pores formed by dissolution of the pore former.

In certain embodiments, the film forming substance comprises gelatin.

In certain embodiments, the gelatin is crosslinked.

In certain embodiments, the degree of crosslinking controls the rate of release of the active agent from the capsule.

In certain embodiments, the gelatin is crosslinked with an aldehyde, e.g., a bifunctional aldehyde, a reducing sugar, or divalent ions. In certain embodiments, the gelatin is crosslinked with divalent ions, without the presence of an aldehyde. In certain embodiments, the gelatin is crosslinked with a reducing sugar without the use of an aldehyde. In some embodiments, when the gelatin is crosslinked with a reducing sugar, then the reducing sugar may form an aldehyde. In certain embodiments, the aldehyde is formaldehyde.

In certain embodiments, the crosslinking is incorporated into the shell composition during manufacture of the capsule. For example, when the capsule is prepared using an encapsulation machine, the crosslinking agent, such as a reducing sugar, can be incorporating into the encapsulation machine to mix with the shell composition.

In certain embodiments, the crosslinking is incorporated into the shell composition post-manufacture of the capsule.

In certain embodiments, the bifunctional aldehyde is formaldehyde.

In certain embodiments, the film forming substance may be a polymer. In some embodiments, the film forming substance may be an animal derived polymer, non-animal derived polymer, or a combination thereof. In some embodiments, the animal derived may include gelatin. The gelatin may include, but is not limited to, Type A gelatin, Type B gelatin, a hide gelatin, a fish gelatin, porcine gelatin and/or a bone gelatin used alone or in-combination. In some embodiments, the gelatin may be Type A medium to high Bloom gelatin. In some embodiments, the gelatin may be Type B medium to high Bloom gelatin. Medium bloom is when the bloom is from about 70 grams to about 160 grams. High bloom is when the bloom is about 175 grams or above, or from about 175 grams to about 300 grams. In some embodiments, the gelatin may be a 250 bloom gelatin. In another embodiment, there is one type of gelatin. In yet another embodiment, there is a combination of at least two types of gelatins. The non-animal derived polymer may include carrageenan.

In some embodiments, the disintegration resistant material may be the same as the film forming substance. In some embodiments, the disintegration resistant material may be different from the film forming substance. In certain embodiments, the disintegration resistant material may include a crosslinked gelatin as described herein.

In certain embodiments, the osmogen comprises polyalkylene oxide, e.g., polyethylene oxide; an osmotic salt, e.g., sodium chloride or potassium chloride; or a sugar alcohol such as xylitol or sorbitol; or a combination of any of the foregoing. In certain embodiments, the active agent can act as an osmogen. In some embodiments, the osmogen may include inorganic salts, carbohydrates, osmotic salt, a polyalkylene oxide or a combination thereof. In some embodiments, the osmogen may include polyethylene oxide, sodium chloride, fructose 3, potassium chloride, sucrose, xylitol, sorbitol, dextrose, citric acid, tartaric acid, mannitol, potassium sulphate, lactose, fumaric acid, adipic acid, lactose-fructose, dextrose-fructose, sucrose-fructose, mannitol-fructose, sodium chloride, fructose, lactose-sucrose, potassium chloride, lactose-dextrose, mannitol-dextrose, dextrose-sucrose, mannitol-sucrose, sucrose, mannitol-lactose, dextrose, potassium sulphate, mannitol, sodium phosphate tribasic-12H₂O, sodium phosphate dibasic-12H₂O, sodium phosphate dibasic-7H₂O, sodium phosphate monobasic-H₂O, sodium phosphate dibasic anhydrous, or a combination thereof.

In certain embodiments, the active agent is an analgesic, an antihistamine, a decongestant, a cough-suppressant or an anti-epileptic.

In certain embodiments, the active agent is acetaminophen or dronabinol.

In certain embodiments, the capsules disclosed herein are contained in a device capable of piercing an orifice into the capsule.

In certain embodiments, the capsule provides a release of the active agent for at least 6 hours, at least 8 hours at least 12 hours or at least 24 hours after oral administration.

In certain embodiments, the invention is directed to a dosing device containing a plurality of capsules as disclosed herein and a piercing element capable of piercing an orifice into a capsule.

In certain embodiments, the capsules are contained in a device in a carousel configuration. In certain embodiments, the device includes a plurality of capsules. In some embodiments, the device may include at least 2 capsules, at least 4 capsules, at least 6 capsules, or at least 8 capsules. In some embodiments, the device may include 2 to 20 capsules, 4 to 18 capsules, 6 to 16 capsules, 8 to 14 capsules, or 10 to 12 capsules. In some embodiments, the device may include 2 capsules, 4 capsules, 6 capsules, 8 capsules, 10 capsules, 12 capsules, 14 capsules, or more.

In certain embodiments, the dosing device as disclosed herein comprises an actuator that upon actuation moves the piercing element from a first nonengaged position relative to a capsule to a second engaged position with the capsule to create an orifice. In some embodiments, the piercing element may include a stylus, drill or a needle. The piercing element may be included various sizes depending on the desired pressure gradient and/or release rate for releasing the liquid fill composition.

In certain embodiments, upon actuation, a pierced capsule is expelled from the device for administration.

In certain embodiments, upon actuation, a proximal capsule is advanced to a first nonengaged position after piercing of the previous capsule.

In certain embodiments, upon actuation, a capsule is advanced to the first nonengaged position and subsequently moved to the second engaged position.

In certain embodiments, the present invention is directed to a method of treating a disease or condition (e.g., pain, fever or epilepsy) comprising forming an orifice in a capsule as disclosed herein and administering the dosage form to a patient in need thereof.

In certain embodiments, the capsule is orally administered within 30 minutes, within 15 minutes, within 5 minutes, within 1 minute, within 30 seconds or immediately after forming the orifice.

In certain embodiments, the present invention is directed to a method of treating a disease or condition comprising administering a capsule as disclosed herein to a patient in need thereof.

In certain embodiments, the present invention is directed to a method of preparing a capsule comprising laser drilling a hole in a capsule as disclosed herein.

In certain embodiments, the method of preparing a capsule includes piercing a hole or orifice in a capsule using a stylus or needle.

The capsule having an orifice or hole may further be sealed to plug the hole or orifice. The sealing may be performed by applying a piezoelectric spray to the capsule. In some embodiments, the sealing may be performed using other suitable methods to plug the hole or orifice. During this process, the hole or orifice is then plugged with a soluble plug. The soluble plug may include material that is soluble in water. Thus, the capsule can be stored and does not need to be immediately given to a patient in need thereof.

The method of preparing may further include enrobing the capsule in a soluble coating. The soluble coating may be made from material that is soluble in water.

The term “condition” or “conditions” refers to those medical conditions that can be treated or prevented by administration to a subject of an effective amount of an active agent.

As used herein, the term “active ingredient” refers to any material that is intended to produce a therapeutic, prophylactic, or other intended effect, whether or not approved by a government agency for that purpose. This term with respect to a specific agent includes the pharmaceutically active agent, and all pharmaceutically acceptable salts, solvates and crystalline forms thereof, where the salts, solvates and crystalline forms are pharmaceutically active.

Any pharmaceutically active ingredient may be used for purposes of the present invention, including both those that are water-soluble and those that are poorly soluble in water. Suitable pharmaceutically active ingredients include, without limitation, analgesics and anti-inflammatory agents, antacids, anthelmintic, anti-arrhythmic agents, anti-bacterial agents, anti-coagulants, anti-depressants, antidiabetics, anti-diarrheal, anti-epileptics, anti-fungal agents, anti-gout agents, anti-hypertensive agents, anti-malarial, anti-migraine agents, anti-muscarinic agents, anti-neoplastic agents and immunosuppressants, anti-protozoal agents, anti-rheumatics, anti-thyroid agents, antivirals, anxiolytics, sedatives, hypnotics and neuroleptics, beta-blockers, cardiac inotropic agents, corticosteroids, cough suppressants, cytotoxics, decongestants, diuretics, enzymes, anti-parkinsonian agents, gastro-intestinal agents, histamine receptor antagonists, lipid regulating agents, local anesthetics, neuromuscular agents, nitrates and anti-anginal agents, nutritional agents, opioid analgesics, oral vaccines, proteins, peptides and recombinant drugs, sex hormones and contraceptives, spermicides, stimulants, and combinations thereof.

In some embodiments, the active pharmaceutical ingredient may be selected, without limitations, from the group consisting of acetaminophen, dronabinol, dabigatran, dronedarone, ticagrelor, iloperidone, ivacaftor, midostaurine, asimadoline, beclomethasone, apremilast, sapacitabine, linsitinib, abiraterone, vitamin D analogs (e.g., calcifediol, calcitriol, paricalcitol, doxercalciferol), COX-2 inhibitors (e.g., celecoxib, valdecoxib, rofecoxib), tacrolimus, testosterone, lubiprostone, pharmaceutically acceptable salts thereof, and combinations thereof.

According to certain embodiments, active agents may include lipid-lowering agents including, but not limited to, statins (e.g., lovastatin, simvastatin, pravastatin, fluvastatin, atorvastatin, rosuvastatin, and pitavastatin), fibrates (e.g, clofibrate, ciprofibrate, bezafibrate, fenofibrate, and gemfibrozil), niacin, bile acid sequestrants, ezetimibe, lomitapide, phytosterols, and the pharmaceutically acceptable salts, hydrates, solvates and prodrugs thereof, mixtures of any of the foregoing, and the like.

Suitable nutraceutical active agents may include, but are not limited to, 5-hydroxytryptophan, acetyl L-carnitine, alpha lipoic acid, alpha-ketoglutarates, bee products, betaine hydrochloride, bovine cartilage, caffeine, cetyl myristoleate, charcoal, chitosan, choline, chondroitin sulfate, coenzyme Q10, collagen, colostrum, creatine, cyanocobalamin (Vitamin 812), dimethylaminoethanol, fumaric acid, germanium sequioxide, glandular products, glucosamine HCl, glucosamine sulfate, hydroxyl methyl butyrate, immunoglobulin, lactic acid, L-Carnitine, liver products, malic acid, maltose-anhydrous, mannose (d-mannose), methyl sulfonyl methane, phytosterols, picolinic acid, pyruvate, red yeast extract, S-adenosylmethionine, selenium yeast, shark cartilage, theobromine, vanadyl sulfate, and yeast.

Suitable nutritional supplement active agents may include vitamins, minerals, fiber, fatty acids, amino acids, herbal supplements or a combination thereof.

Suitable vitamin active agents may include, but are not limited to, the following: ascorbic acid (Vitamin C), B vitamins, biotin, fat soluble vitamins, folic acid, hydroxycitric acid, inositol, mineral ascorbates, mixed tocopherols, niacin (Vitamin B3), orotic acid, para-aminobenzoic acid, panthothenates, panthothenic acid (Vitamin B5), pyridoxine hydrochloride (Vitamin B6), riboflavin (Vitamin B2), synthetic vitamins, thiamine (Vitamin B1), tocotrienols, vitamin A, vitamin D, vitamin E, vitamin F, vitamin K, vitamin oils and oil soluble vitamins.

Suitable herbal supplement active agents may include, but are not limited to, the following: arnica, bilberry, black cohosh, cat's claw, chamomile, echinacea, evening primrose oil, fenugreek, flaxseed, feverfew, garlic, ginger root, ginko biloba, ginseng, goldenrod, hawthorn, kava-kava, licorice, milk thistle, psyllium, rauowolfia, senna, soybean, St. John's wort, saw palmetto, turmeric, valerian.

Examples of other possible active agents include, but are not limited to, antihistamines (e.g., ranitidine, dimenhydrinate, diphenhydramine, chlorpheniramine and dexchlorpheniramine maleate), non-steroidal anti-inflammatory agents (e.g., aspirin, celecoxib, Cox-2 inhibitors, diclofenac, benoxaprofen, flurbiprofen, fenoprofen, flubufen, indoprofen, piroprofen, carprofen, oxaprozin, pramoprofen, muroprofen, trioxaprofen, suprofen, aminoprofen, fluprofen, bucloxic acid, indomethacin, sulindac, zomepirac, tiopinac, zidometacin, acemetacin, fentiazac, clidanac, oxpinac, meclofenamic acid, flufenamic acid, niflumic acid, tolfenamic acid, diflurisal, flufenisal, piroxicam, sudoxicam, isoxicam, aceclofenac, aloxiprin, azapropazone, benorilate, bromfenac, carprofen, choline magnesium salicylate, diflunisal, etodolac, etoricoxib, faislamine, fenbufen, fenoprofen, flurbiprofen, ibuprofen, indometacin, ketoprofen, ketorolac, lornoxicam, loxoprofen, meloxicam, mefenamic acid, metamizole, methyl salicylate, magnesium salicylate, nabumetone, naproxen, nimesulide, oxyphenbutazone, parecoxib, phenylbutazone, salicyl salicylate, sulindac, sulfinpyrazone, tenoxicam, tiaprofenic acid, tolmetin. pharmaceutically acceptable salts thereof and mixtures thereof) and acetaminophen, anti-emetics (e.g., metoclopramide, methylnaltrexone), anti-epileptics (e.g., phenyloin, meprobmate and nitrazepam), vasodilators (e.g., nifedipine, papaverine, diltiazem and nicardipine), anti-tussive agents and expectorants (e.g. codeine phosphate), anti-asthmatics (e.g. theophylline), antacids, anti-spasmodics (e.g. atropine, scopolamine), antidiabetics (e.g., insulin), diuretics (e.g., ethacrynic acid, bendrofluthiazide), anti-hypotensives (e.g., propranolol, clonidine), antihypertensives (e.g., clonidine, methyldopa), bronchodilatiors (e.g., albuterol), steroids (e.g., hydrocortisone, triamcinolone, prednisone), antibiotics (e.g., tetracycline), antihemorrhoidals, hypnotics, psychotropics, antidiarrheals, mucolytics, sedatives, decongestants (e.g. pseudoephedrine), laxatives, vitamins, stimulants (including appetite suppressants such as phenylpropanolamine) and cannabinoids, as well as pharmaceutically acceptable salts, hydrates, solvates, and prodrugs thereof.

The active agent that may also be a benzodiazepine, barbiturate, stimulants, or mixtures thereof. The term “benzodiazepines” refers to a benzodiazepine and drugs that are derivatives of a benzodiazepine that are able to depress the central nervous system. Benzodiazepines include, but are not limited to, alprazolam, bromazepam, chlordiazepoxide, clorazepate, diazepam, estazolam, flurazepam, halazepam, ketazolam, lorazepam, nitrazepam, oxazepam, prazepam, quazepam, temazepam, triazolam, methylphenidate as well as pharmaceutically acceptable salts, hydrates, solvates, prodrugs and mixtures thereof. Benzodiazepine antagonists that can be used as active agent include, but are not limited to, flumazenil as well as pharmaceutically acceptable salts, hydrates, solvates and mixtures thereof.

The term “barbiturates” refers to sedative-hypnotic drugs derived from barbituric acid (2, 4, 6,-trioxohexahydropyrimidine). Barbiturates include, but are not limited to, amobarbital, aprobarbotal, butabarbital, butalbital, methohexital, mephobarbital, metharbital, pentobarbital, phenobarbital, secobarbital as well as pharmaceutically acceptable salts, hydrates, solvates, prodrugs, and mixtures thereof. Barbiturate antagonists that can be used as active agent include, but are not limited to, amphetamines as well as pharmaceutically acceptable salts, hydrates, solvates and mixtures thereof.

The term “stimulants” includes, but is not limited to, amphetamines such as dextroamphetamine resin complex, dextroamphetamine, methamphetamine, methylphenidate, as well as pharmaceutically acceptable salts, hydrates, and solvates and mixtures thereof. Stimulant antagonists that can be used as active agent include, but are not limited to, benzodiazepines, as well as pharmaceutically acceptable salts, hydrates, solvates and mixtures thereof.

The dosage forms according to the disclosure include various active agents and their pharmaceutically acceptable salts thereof. Pharmaceutically acceptable salts include, but are not limited to, inorganic acid salts such as hydrochloride, hydrobromide, sulfate, phosphate and the like; organic acid salts such as formate, acetate, trifluoroacetate, maleate, tartrate and the like; sulfonates such as methanesulfonate, benzenesulfonate, p-toluenesulfonate, and the like; amino acid salts such as arginate, asparginate, glutamate and the like, and metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N, N′-dibenzylethylenediamine salt and the like.

As used herein, the terms “therapeutically effective” and an “effective amount” refer to the amount of active agent or the rate at which it is administered which is needed to produce a desired therapeutic result.

As used herein, “shell” or “shell composition” refers to the shell of a softgel capsule which encapsulates a fill material.

All references to wt % throughout the specifications and the claims refer to the weight of the component in reference to the weight of the entire composition and may also be designated as w/w.

As used herein, “fill material” or “fill” refers to the composition that is encapsulated by the capsule shell and contains at least one pharmaceutically active ingredient.

As used herein, “about” refers to any values that are within a variation of ±10%, such that “about 10” would include from 9 to 11. As used herein, “a,” “an,” or “the” refers to one or more, unless otherwise specified. Thus, for example, reference to “an excipient” includes a single excipient as well as a mixture of two or more different excipients, and the like.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

The use of any and all examples, or exemplary language (e.g., “such as”) provided herein, is intended merely to illuminate certain materials and methods and does not pose a limitation on scope. No language in the specification should be construed as indicating any non-claimed element as essential to the practice of the disclosed materials and methods.

In addition to the osmogen, other suitable fill materials include flavoring agents, sweetening agents, coloring agents and fillers or other pharmaceutically acceptable excipients or additives such as synthetic dyes and mineral oxides. Suitable amounts of pharmaceutically active ingredient and pharmaceutically acceptable excipients can be readily determined by one of ordinary skill in the art.

In an embodiment, the gelatin in the shell composition may include Type A gelatin, Type B gelatin, a hide gelatin and/or a bone gelatin used alone or in combination. In one embodiment, the gelatin is a 250 bloom gelatin (a high molecular weight gelatin to form more crosslinks, bloom does not necessarily correlate with molecular weight). In another embodiment, there is only one type of gelatin. In yet another embodiment, the gelatin is a combination of at least two types of gelatins. In an embodiment, the amount of gelatin in the shell composition is about 40 wt % to about 80 wt %, more preferably from about 45 wt % to about 75 wt %, and most preferably from about 50 wt % to about 70 wt %.

In one embodiment, the capsule shell composition comprises hydroxypropylmethyl cellulose (“HPMC”). In an embodiment, the amount of cellulose derivative (e.g., methyl cellulose or HPMC) in the capsule shell composition is about 0.15 wt % to about 4.0 wt %, more preferably from about 0.20 wt % to about 2.0 wt %, and most preferably from about 0.25 wt % to about 1.4 wt %. In some embodiments, the capsule shell composition may comprise HPMC, methyl cellulose (MC), hydroxypropylcellulose (HPC), or combinations thereof. The cellulose derivative may be added to the capsule shell to mitigate potential reduction in gel strength. The concentration of cellulose derivative in the shell composition may be in an effective amount to improve the gel strength but not so high that it would interfere with the seal.

In some embodiments, the shell composition may comprise pectin, e.g., a low methoxy pectin. In an embodiment, the low methoxy pectin may be LM Pectin (P-25), LM Pectin (445C), LM Pectin (100C) or a combination thereof. The addition of pectin contributes to the nature of the dosage form. However, too much pectin in the dosage form may reduce the gel strength of the softgel capsule which may in turn adversely affect the sealability of the softgel capsule. Therefore, pectin may be added to the dosage form at a concentration that is sufficiently high to form a dosage form and at the same time is sufficiently low to mitigate the reduction in gel strength. In an embodiment, an amount of low methoxy pectin in the shell composition is about 2 wt % to about 20 wt %, from about 3 wt % to about 15 wt %, from about 3 wt % to about 5.5 wt %, and from about 5 wt % to about 10 wt %. The degree of esterification of the pectin incorporated in the shell composition may be lower than about 50%, or may range from about 10% to about 50%, from about 20% to about 40%, or from about 25% to about 35%. In certain embodiments, the pectin can be present in combination with an acrylic polymer (e.g., about 10% to about 30%) such as N, N-dimethylaminoethyl methacrylate with methylmethacrylate and butylmethacrylate (Eudragit EPO®). In such an embodiment, the pectin prevents solubility under acid conditions and the acrylic polymer prevents solubility in basic conditions.

In an embodiment, the plasticizer in the shell composition may include glycerol, glycerin, sorbitol and combinations thereof. Other suitable plasticizers may include, but not be limited to, sugar alcohol plasticizer such as isomalt, maltitol, xylitol, erythritol, adonitol, dulcitol, pentaerythritol, or mannitol; or polyol plasticizer such as diglycerin, ethylene glycol, diethylene glycol, triethyleneglycol, tetraethylene glycol, dipropylene glycol, a polyethylene glycol up to 10,000 MW, neopentyl glycol, propylene glycol, 1,3-propanediol, 2-methyl-1,3-propanediol, trimethylolpropane, a polyether polyol, ethanol amines; and mixtures thereof. Other exemplary plasticizers may also include, without limitations, low molecular weight polymers, oligomers, copolymers, oils, small organic molecules, low molecular weight polyols having aliphatic hydroxyls, ester-type plasticizers, glycol ethers, poly(propylene glycol), multi-block polymers, single block polymers, citrate ester-type plasticizers, and triacetin. Such plasticizers may include 1,2-butylene glycol, 2,3-butylene glycol, styrene glycol, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutyl sebacate, acetyltributylcitrate, triethyl citrate, glyceryl monostearate, polysorbate 80, acetyl triethyl citrate, tributyl citrate and allyl glycolate, and mixtures thereof.

In an embodiment, the amount of plasticizer in the shell composition is about 15 wt % to about 40 wt %, more preferably from about 20 wt % to about 35 wt %, and most preferably from about 25 wt % to about 30 wt %.

In some embodiments, the shell composition may include a soluble layer in combination with an insoluble coating. In some embodiments, the shell composition includes an insoluble layer. In some embodiments the shell composition may include a soluble layer in contact with the fill material, or an insoluble layer may be in contact with the fill material. In some embodiments, the soluble layer may include a soluble polymer and additional excipients as described herein. In some embodiments, the insoluble layer may include a disintegration resistant material, a basic resistant material, or combination thereof and an additional excipient as described herein.

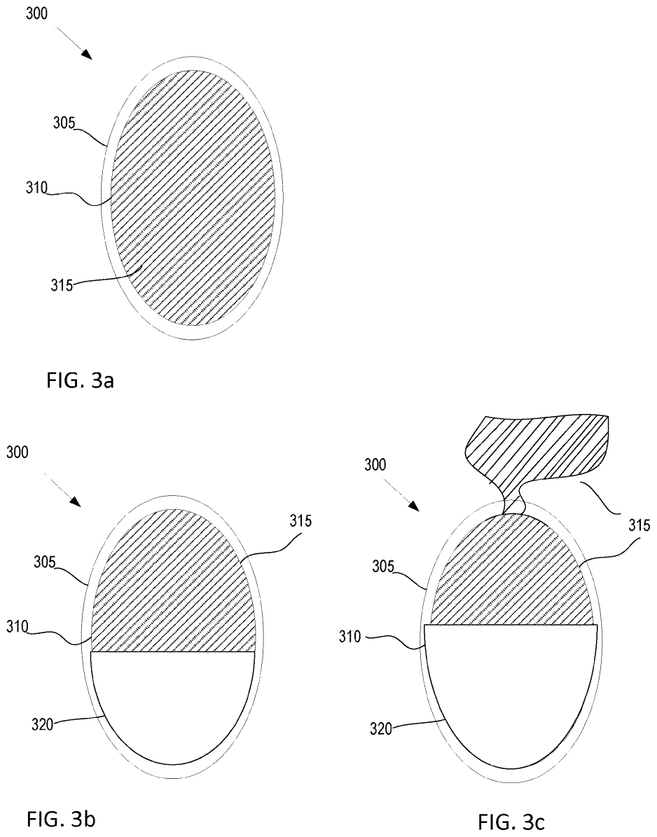

In some embodiments, the capsule as described herein may include either an orifice in a shell composition having an insoluble layer, or an orifice in a shell composition have a soluble layer in combination with an insoluble coating. The capsule is then exposed to water, such that the water may imbibe through the shell composition and cause an osmotic pressure to release the fill material through the orifice. In some embodiments, the water may mix with the fill material to dilute the fill material present in the capsule, as the active agent including the osmogen releases at a slow release rate through the orifice. It is noted that depending on the active agent, it may be undesired for the water to mix with the fill material. Therefore, to avoid mixing with the fill material, a separate osmogen layer may be present in the capsule and act as a piston to drive the fill material through the orifice as is illustrated in FIGS. 3b and 3c.

In other embodiments the shell composition may optionally comprise additional agents such as coloring agents, flavorings agents, sweetening agents, fillers, antioxidants, diluents, pH modifiers or other pharmaceutically acceptable excipients or additives such as synthetic dyes and mineral oxides.

Exemplary suitable coloring agents may include, but not be limited to, colors such as e.g., white, black, yellow, blue, green, pink, red, orange, violet, indigo, and brown. In specific embodiments, the color of the dosage form can indicate the contents (e.g., one or more active ingredients) contained therein.

Exemplary suitable flavoring agents may include, but not be limited to, “flavor extract” obtained by extracting a part of a raw material, e.g., animal or plant material, often by using a solvent such as ethanol or water; natural essences obtained by extracting essential oils from the blossoms, fruit, roots, etc., or from the whole plants.

Additional exemplary flavoring agents that may be in the dosage form may include, but not be limited to, breath freshening compounds like menthol, spearmint, and cinnamon, coffee beans, other flavors or fragrances such as fruit flavors (e.g., cherry, orange, grape, etc.), especially those used for oral hygiene, as well as actives used in dental and oral cleansing such as quaternary ammonium bases. The effect of flavors may be enhanced using flavor enhancers like tartaric acid, citric acid, vanillin, or the like.

Exemplary sweetening agents may include, but not be limited to, one or more artificial sweeteners, one or more natural sweeteners, or a combination thereof. Artificial sweeteners include, e.g., acesulfame and its various salts such as the potassium salt (available as Sunett®), alitame, aspartame (available as NutraSweet® and Equal®), salt of aspartame-acesulfame (available as Twinsweet®), neohesperidin dihydrochalcone, naringin dihydrochalcone, dihydrochalcone compounds, neotame, sodium cyclamate, saccharin and its various salts such as the sodium salt (available as Sweet'N Low®), stevia, chloro derivatives of sucrose such as sucralose (available as Kaltame® and Splenda®), and mogrosides. Natural sweeteners include, e.g., glucose, dextrose, invert sugar, fructose, sucrose, glycyrrhizin; monoammonium glycyrrhizinate (sold under the trade name MagnaSweet®); Stevia rebaudiana (Stevioside), natural intensive sweeteners, such as Lo Han Kuo, polyols such as sorbitol, mannitol, xylitol, erythritol, and the like. Some flavors may also be utilized as crosslinking agents.

Encapsulation of the fill material can be accomplished in any conventional manner. As an example, a rotary die encapsulation may be used.

According to an embodiment, a softgel capsule is prepared by the process comprising the steps of: (a) preparing the fill material, said fill material comprising at least one pharmaceutically active ingredient and an osmogen; and (b) encapsulating the fill material of step (a) in a semi-permeable shell composition as disclosed herein.

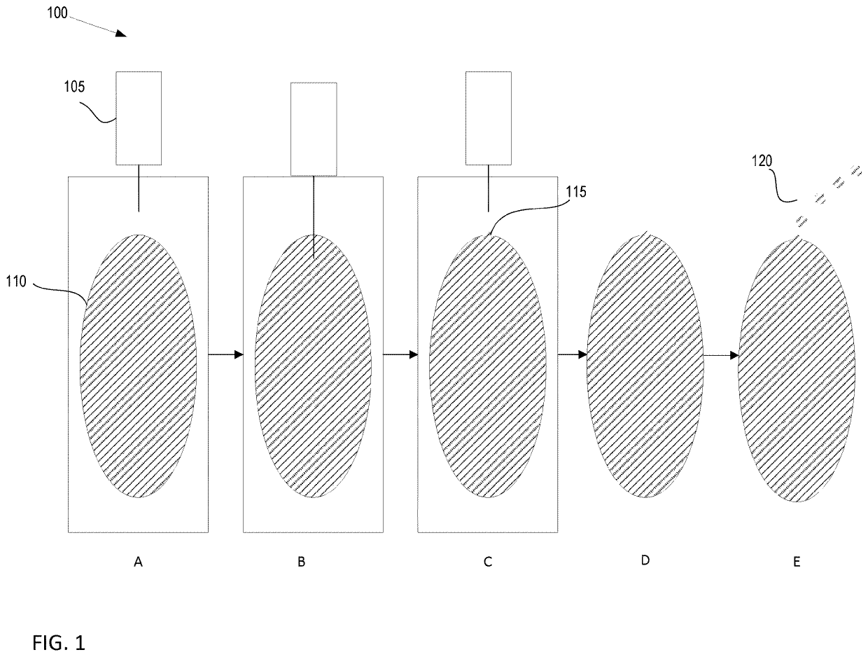

Referring now to the Figures, FIG. 1 shows a progression of a liquid filled capsule in an embodiment of the present disclosure 100. As can be seen in block A of FIG. 1, a capsule 110 was prepared having a fill material and a shell composition. In some embodiments, the shell composition may include a film forming material and/or a disintegration resistant material as described herein. The shell composition may include insoluble or disintegration resistant materials, or could be coated with a substance to provide this performance. For example, the disintegration resistant shell may include crosslinked gelatin or other polymers, such as alginate or carrageenan. In some embodiments, the film forming material may include a polymer, gelatin or a combination thereof. In some embodiments, the fill material may be hypoosmotic, which will cause water to migrate into the capsule. In some embodiments, the fill material may be not hypoosmotic, such as oils, where the fill material will not be diluted when water migrates into an interior of the capsule. When the fill material is not hypoosmotic, an osmogen is included in the fill material. Alternatively, a two component fill system may be used in the capsule, where one end is filled with a fill material and the other end is filled with an osmogen. Thus, when pressure is applied, then the osmogen can move through the capsule to push the fill material out of the capsule.

In block B, the capsule was pierced using a stylus 1085. It should be understood that the capsule may be pierced using a different tool, such as a needle or a laser. The stylus may be a variety of sizes depending on target delivery of the dosage. In some embodiments, the stylus may have a size of 15 gauge to about 25 gauge. In block C, the stylus 105 is removed from the capsule 110 to create an orifice 115 in the capsule. It is understood that the size of the orifice 115 corresponds to the size of the stylus.

After being pierced, the capsule 110 is then placed in water in blocked D. As can be seen in block E, when the capsule 110 is placed in water, the fill material 120 migrates outside of the capsule through the orifice. For example, the water can enter the interior of the capsule, which forces the fill material 120 out of the capsule 110. Thus, a period of relatively constant delivery of the fill material 102 will occur and slowly taper as the contents of the capsule become diluted.

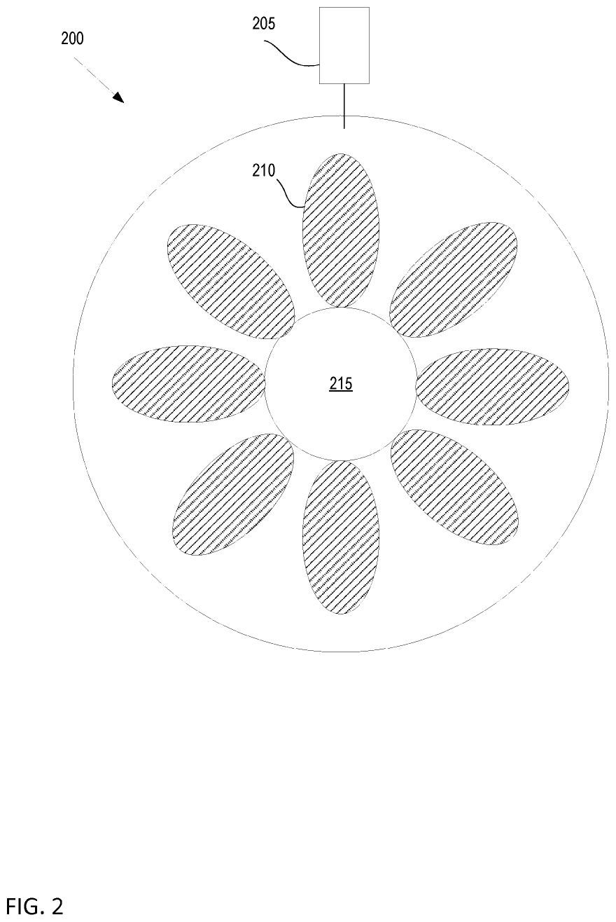

In FIG. 2, a dispensing system 200 according to an embodiment of the present disclosure is illustrated. In FIGS. 2, 8 capsules 210 are loaded into a carousel 215. The carousel 215 can be rotated to a position that aligns with the stylus 205. The stylus 205 can be depressed, which then pierced the capsule 210. The capsule 210 was then ejected from the carousel 215. It is believed that such dispensing system 200 can provide a convenient means to deliver a sustained delivery system of a liquid filled system. That is, the dispensing system 200 can assure proper use by the patient as well.

In FIG. 3a, a cross section of a capsule 300 in accordance with an embodiment of the present disclosure is provided. The capsule 300 includes a fill material 315. The fill material 315 includes an osmogen. In some embodiments, the osmogen may include inorganic salts, carbohydrates, osmotic salt, a polyalkylene oxide or a combination thereof. In some embodiments, the osmogen may include polyethylene oxide, sodium chloride, fructose 3, potassium chloride, sucrose, xylitol, sorbitol, dextrose, citric acid, tartaric acid, mannitol, potassium sulphate, lactose, fumaric acid, adipic acid, lactose-fructose, dextrose-fructose, sucrose-fructose, mannitol-fructose, sodium chloride, fructose, lactose-sucrose, potassium chloride, lactose-dextrose, mannitol-dextrose, dextrose-sucrose, mannitol-sucrose, sucrose, mannitol-lactose, dextrose, potassium sulphate, mannitol, sodium phosphate tribasic-12H₂O, sodium phosphate dibasic-12H₂O, sodium phosphate dibasic-7H₂O, sodium phosphate monobasic-H₂O, sodium phosphate dibasic anhydrous, or a combination thereof. In some embodiments, the fill material further includes an active agent as described herein.

The capsule 300 further includes a shell composition including a soluble shell 310 that encapsulates the fill material 315. The capsule 300 also includes an insoluble coating 305 that is in direct contact with the soluble shell 310. It should be understood that there is no gap in the insoluble coating 305 and soluble shell 310. In some embodiments, the soluble shell 310 may include a polymer, gelatin, a plasticizer, pectin, dextrose or a combination thereof. In some embodiments, the polymer may include hydroxymethyl cellulose. In some embodiments, the pectin may include amidated pectin or non-amidated pectin. In some embodiments, the plasticizer may include glycerol, glycerin, sorbitol, a polyethylene sorbitan monooleate or a combination thereof. Other suitable plasticizers may include, but not be limited to, sugar alcohol plasticizer such as isomalt, maltitol, xylitol, erythritol, adonitol, dulcitol, pentaerythritol, or mannitol; or polyol plasticizer such as diglycerin, ethylene glycol, diethylene glycol, triethyleneglycol, tetraethylene glycol, dipropylene glycol, a polyethylene glycol up to 10,000 MW, neopentyl glycol, propylene glycol, 1,3-propanediol, 2-methyl-1,3-propanediol, trimethylolpropane, a polyether polyol, ethanol amines; and mixtures thereof. Other exemplary plasticizers may also include, without limitations, low molecular weight polymers, oligomers, copolymers, oils, small organic molecules, low molecular weight polyols having aliphatic hydroxyls, ester-type plasticizers, glycol ethers, poly(propylene glycol), multi-block polymers, single block polymers, citrate ester-type plasticizers, and triacetin. Such plasticizers may include 1,2-butylene glycol, 2,3-butylene glycol, styrene glycol, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutyl sebacate, acetyltributylcitrate, triethyl citrate, glyceryl monostearate, polysorbate 80, acetyl triethyl citrate, tributyl citrate and allyl glycolate, and mixtures thereof. In another embodiment, the soluble shell 310 may not be present, and only the insoluble shell 305 is present, where the insoluble shell 305 is encapsulates the fill material 315.

In some embodiments, the insoluble coating may include a crosslinked gelatin as described herein. The crosslinked gelatin may be formed using a reducing sugar, an aldehyde, divalent ions, or an amine polymer. The amine polymer, such as polylysine, can crosslink with gelatin in the presence of an aldehyde, such as formaldehyde. In some embodiments, the gelatin can crosslink in the presence of the reducing sugar without the use of an aldehyde. In some embodiments, the insoluble coating may include a disintegration resistant material as described herein.

In FIGS. 3b and 3c, an alternate embodiment of the capsule 300 is provided. In FIGS. 3b and 3c, the shell composition can include a soluble layer 310 in combination with an insoluble layer 305. In some embodiments, the shell composition only includes an insoluble layer 305. In the capsule 300 of FIGS. 3b and 3c, the fill material 315 is prepared in combination with an osmogen 320. When the capsule 300 is exposed to water, pressure is created which causes the osmogen 320 to push the fill material 315 to be released from the capsule 300 as described throughout the present disclosure.

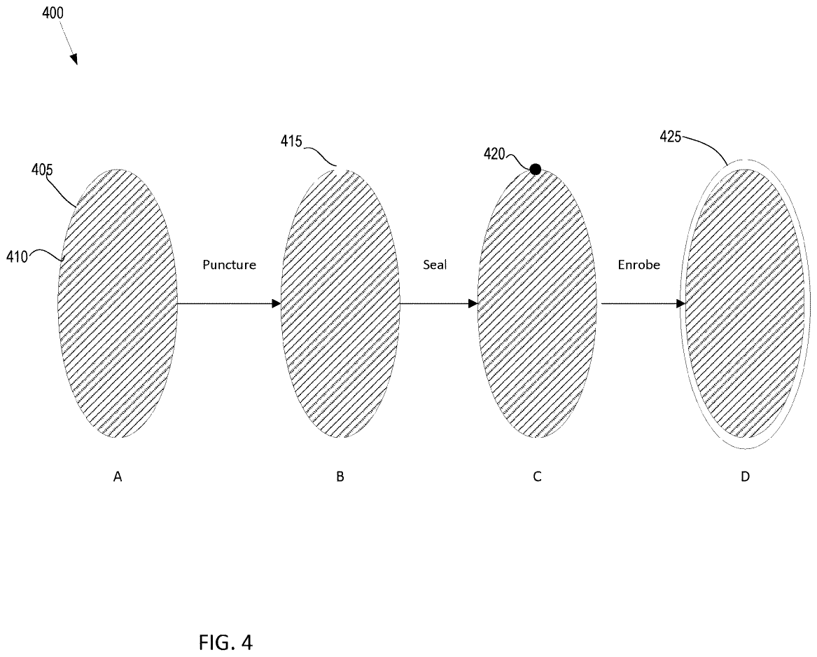

Referring to FIG. 4, in block A, a liquid filled capsule 405 is prepared having a fill material 410, which can be the fill material described in referenced to FIG. 1 or any fill material described herein. The liquid filled capsule 405 further includes a shell composition, which can be the shell composition described in relation to FIG. 1. The liquid filled capsule 405 is punctured between block A and B to form a hole or orifice 415 in the shell composition. The capsule 405 may be punctured using a laser, a drill, a stylus or a needle to form the orifice 415. The orifice 415 may be formed to have a diameter size of about 600 μm to about 1 mm, about 700 μm to about 900 μm, or about 750 μm to about 850 μm. After forming the orifice 415, it is then sealed in block C with a plug 420. To plug the orifice, a piezoelectric spray is applied to the capsule. The spray then forms a plug 420 so that the capsule 405 can be stored until the capsule is exposed to water. The capsule 405 is optionally further enrobed in an additional soluble coating 425 in block D. In some embodiments, the liquid filled capsule 405 may not undergo a sealing stop of block C, but will be enrobed in an additional soluble coating 425 of block D. Thus, the capsule 405 can be plugged, enrobed, or plugged and enrobed.

As understood herein, the term “enrobe” may be interchanged with “coat” or “coating” and refers to an enclosing layer around a capsule. The enclosing layer or coating 425 is soluble in accordance with the present disclosure. The coating 425 is in direct contact with the plug 420 and shell composition of the capsule 405. Thus, the capsule 405 can be stored and later exposed to water which will cause the fill material to be slowly released as discussed herein. If the sealing step of block C is not performed, then the enrobing process of block D will continuously be performed as the capsule is punctured to prevent excessive leaking prior to enrobing the capsule.

### EXAMPLES

Specific embodiments of the invention will now be demonstrated by reference to the following examples. It should be understood that these examples are disclosed solely by way of illustrating the invention and should not be taken in any way to limit the scope of the present invention.

### Example 1

Softgel capsules were prepared with a capsule shell including gelatin, sorbitol sorbitan, glycerin and water and a fill material including acetaminophen, povidone, polyethylene glycol 600, potassium acetate and water.

Crosslinking of the shell composition was performed by incubating the capsules in glass desiccator, equilibrated with Formaldehyde 37% in aqueous solution for 24 hours before placing the capsules, 80 capsules were transferred into the desiccator and placed in monolayer in a plate. At 3, 6, 12 and 24 hours, 20 capsules were removed from desiccator and placed in 60-cc glass jar with cap.

Disintegration tests were performed with a capsule of each crosslinking group placed in 800 ml Simulated Gastric Fluid (SGF, pH 1.6), preheated to 37.4° C. The release of acetaminophen (“APAP”) was measured in real time using Pion FiberOptic technology. After 30 minutes, concentrated Fasted Simulated intestinal Fluid (FaSSIF) was added to final pH of approximately 6. The release was measures for 5 to 12 hours from the beginning of the experiment.

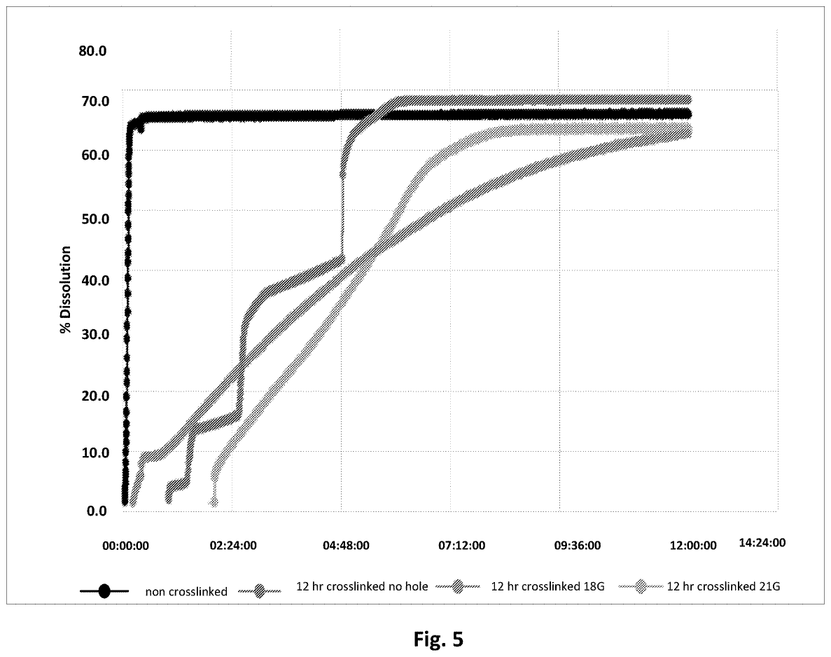

FIG. 5 shows the percent release over time of non-crosslinked capsules, 12 hour crosslinked capsules with no orifice, 12 hour crosslinked capsules with an orifice by piercing with an 18G needle, and 12 hour crosslinked capsules with an orifice by piercing with a 21G needle. As can be seen in FIG. 5, the non-crosslinked capsules showed immediate release. The capsules with an orifice formed with a 18 gauge needle that was crosslinked for 12 hours showed a continuous release profile. The capsules with an orifice formed with a 21 gauge needle that was crosslinked for 12 hours showed a pulsatile type release profile because of the smaller orifice and the high viscosity of the fill material, i.e. internal pressure builds and cause a release pulse which relieves the pressure, followed by pressure build and a second pulse, etc. The capsules that were crosslinked for 12 hours with no hole shows a build up of internal pressure followed by rupture of the capsule which creates a large hole and continuous release profile.

In summary, the inventors believe that the release profile will be impacted by the osmogen, degree of water permeability (e.g., crosslinking), orifice size, elasticity of the shell and viscosity of the fill.

1. A capsule comprising:
a liquid fill material comprising an osmogen and an active agent; and
a semi-permeable disintegration resistant shell composition.

2. The capsule of claim 1, wherein the shell comprises a film forming substance, a disintegration resistant material, or a combination thereof.

3. The capsule of claim 2, wherein the film forming substance and the disintegration resistant material are inter-dispersed.

4. The capsule of claim 2, wherein the shell comprises an inner layer comprising the film forming substance and an outer layer comprising the disintegration resistant material.

5. The capsule of claim 1, wherein the capsule is a hard capsule.

6. The capsule of claim 5, wherein the capsule is a two piece hard capsule.

7. The capsule of claim 1, wherein the capsule is a softgel capsule.

8. The capsule of claim 1, wherein the semi-permeable shell composition is permeable to the passage of water and impervious to the passage of the active agent.

9. The capsule of claim 1, wherein the semi-permeable shell composition comprises at least one orifice.

10. (canceled)

11. The capsule of claim 9, further comprising a soluble plug filling or covering the orifice.

12. The capsule of claim 9, further comprising an additional soluble coating enrobing the shell composition and the orifice.

13. The capsule of claim 1, further comprising an expanding component in the capsule adjacent to the fill material.

14. The capsule of claim 1, further comprising an expanding component layered around the fill material.

15. The capsule of claim 13, wherein the fill material is adjacent to an orifice.

16.-19 (canceled)

20. The capsule of claim 1, wherein the film forming substance comprises gelatin and the shell further comprises an enteric material and a basic resistant polymer.

21. (canceled)

22. The capsule of claim 1, wherein the shell comprises an enteric material and a basic resistant polymer.

23.-30. (canceled)

31. The capsule of claim 1, wherein the osmogen comprises polyalkylene oxide; an osmotic salt; a sugar alcohol; or a combination of any of the foregoing.

32.-35 (canceled)

36. The capsule of claim 1, wherein the capsule is contained in a device capable of piercing an orifice into the capsule.

37. (canceled)

38. A dosing device containing a plurality of capsules according to claim 1 and a piercing element capable of piercing an orifice into a capsule.

39.-46. (canceled)

47. A method of preparing a capsule comprising laser drilling a hole in a capsule according to claim 1.
